(19) United States
(12) Patent Application Publication
(10) Pub. No.: US 2022/0107428 A1
(43) Pub. Date: Apr. 7, 2022

(54) DEVICE FOR IMAGING, RADIATION IMAGING SYSTEM, AND RECORDING MEDIUM

(71) Applicant: Konica Minolta, Inc., Tokyo (JP)

(72) Inventor: Taketoh MURAOKA, Tokyo (JP)

(21) Appl. No.: 17/490,675

(22) Filed: Sep. 30, 2021

(30) Foreign Application Priority Data
Oct. 5, 2020 (JP) 2020-168396

(51) Int. Cl. G01T 1/161

(52) U.S. Cl. CPC G01T 1/161

(57) ABSTRACT

A device for imaging is used for dynamic imaging that repeatedly generates a plurality of frames and has a hardware processor. The hardware processor acquires rhythm information from a storage for storing the rhythm information that defines a rhythm in order to cause a subject to recognize a start timing, a switch timing, or a stop timing of a predetermined periodic motion and notifies the subject of the rhythm based on the acquired rhythm information at least one of periods before starting generation of a frame and during repeating of the generation of the frame.

### CROSS-REFERENCE TO RELATED APPLICATIONS

The entire disclosure of Japanese Patent Application No. 2020-168396 filed on Oct. 5, 2020 is incorporated herein by reference in its entirety.

### BACKGROUND

### Technological Field

The present invention relates to a device for imaging, a radiation imaging system, and a recording medium.

### Description of the Related Art

Various techniques for instructing a subject to start, switch, stop, and the like the periodic motions (breathing, bending and stretching of joints, and the like) that are desired to be performed at the time of imaging when performing dynamic imaging that repeatedly generates a plurality of frames have been proposed.

For example, JP 2019-201929 A discloses a radiation imaging system including a storage that stores an imaging guide pattern for instructing a subject to perform a predetermined motion at the time of imaging by the imager in association with a predetermined word included in an imaging order, an acquisitor that acquires the imaging order for imaging by the imager, a selector that extracts the predetermined word from the acquired imaging order and selects the imaging guide pattern associated with the extracted predetermined word from the imaging guide pattern stored in the storage, and an instructor that instructs the subject to perform the predetermined motion based on the selected imaging guide pattern.

However, in the imaging guide pattern in the related art as described in JP 2019-201929 A, the subject is instructed in a language. For this reason, in the dynamic imaging in the related art performed while instructing the motion, it is necessary to prepare an imaging guide pattern having different contents according to the periodic motion to be imaged.

For example, in the case of imaging the breathing of a subject, it was necessary to prepare a pattern for deep breathing, a pattern for natural breathing, a pattern for labored breathing, and the like.

In particular, in the case of imaging the bending and stretching of bones and joints, it was necessary to prepare different imaging guide patterns depending on not only the motion but also the imaging site, such as a pattern of instructing the expansion and contraction of the legs and arms, a pattern of instructing turning, a pattern of instructing flexion, and a pattern of instructing the anterior-posterior bending of the waist.

In addition, as the number of types of imaging guide patterns used in this manner increases, the operation for selecting the imaging guide pattern becomes complicated.

In addition, in recent years, the types of periodic motions to be imaged have increased.

In order to make the system compatible with imaging of new periodic motions, for example, a labor of a serviceman of the system manufacturer going to the installation location of the system and adding data of an imaging guide pattern for instructing new periodic motions occurs.

In addition, as the data of the imaging guide pattern increases, there is a possibility that the data capacity of the storage of the system becomes tight, or the process for switching the pattern becomes complicated.

### SUMMARY

The invention has been made in view of the above problems, and an object of the invention is that, in dynamic imaging which is performed while instructing a subject to start, switch, or stop a predetermined periodic motion, instruction of a plurality of periodic motions having different contents is enabled to be performed without a device configuration and an operation by a user being complicated.

In order to solve the above problems, according to one aspect of the invention, there is provided a device for imaging used for dynamic imaging that repeatedly generates a plurality of frames including a hardware processor, in which the hardware processor acquires rhythm information from a storage for storing the rhythm information that defines a rhythm in order to cause a subject to recognize a start timing, a switch timing, or a stop timing of a predetermined periodic motion, and in which the hardware processor notifies the subject of the rhythm based on the acquired rhythm information before starting generation of a frame and during at least one of several periods of repeating the generation of the frame.

In order to solve the above problems, according to another aspect of the invention, there is provided a radiation imaging system including: a storage that stores rhythm information that defines a rhythm for a subject to recognize a start timing, a switch timing, or a stop timing of a predetermined periodic motion; a hardware processor that acquires the rhythm information from the storage; an image generator that repeatedly generates frames constituting dynamic image; and a notificator that notifies the subject of the rhythm based on the rhythm information acquired by the hardware processor before starting generation of a frame and during at least one of several periods of repeating the generation of the frame by the image generator.

According to still another aspect of the invention, there is provided a computer-readable non-temporary recording medium containing a program, in which the program causes a hardware processor of a device for imaging used for dynamic imaging that repeatedly generates a plurality of frames to execute: an acquisition process of acquiring rhythm information from a storage for storing the rhythm information that defines a rhythm in order to cause a subject to recognize a start timing, a switch timing, or a stop timing of a predetermined periodic motion; and a notification process for notifying the subject of the rhythm based on the rhythm information acquired in the acquisition process before starting generation of a frame and during at least one of several periods of repeating the generation of the frame.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram showing a radiation imaging system according to an embodiment of the invention;

FIG. 2 is a block diagram showing a device for imaging included in the radiation imaging system of FIG. 1;

FIG. 3 is a flowchart showing an example of a flow of a motion instruction process executed by the device for imaging of FIG. 2;

FIG. 4 is a flowchart showing another example of the flow of the motion instruction process executed by the device for imaging of FIG. 2; and

FIG. 5 is a diagram showing correspondence between operations of the device for imaging of FIG. 2 and generated frames.

### DETAILED DESCRIPTION OF THE EMBODIMENTS

Hereinafter, embodiments of the invention will be described with reference to the drawings. However, the technical scope of the invention is not limited to the following embodiments and illustrated examples.

[1. Radiation Imaging System]

First, a schematic configuration of a radiation imaging system (hereinafter referred to as a system 100) according to the present embodiment will be described.

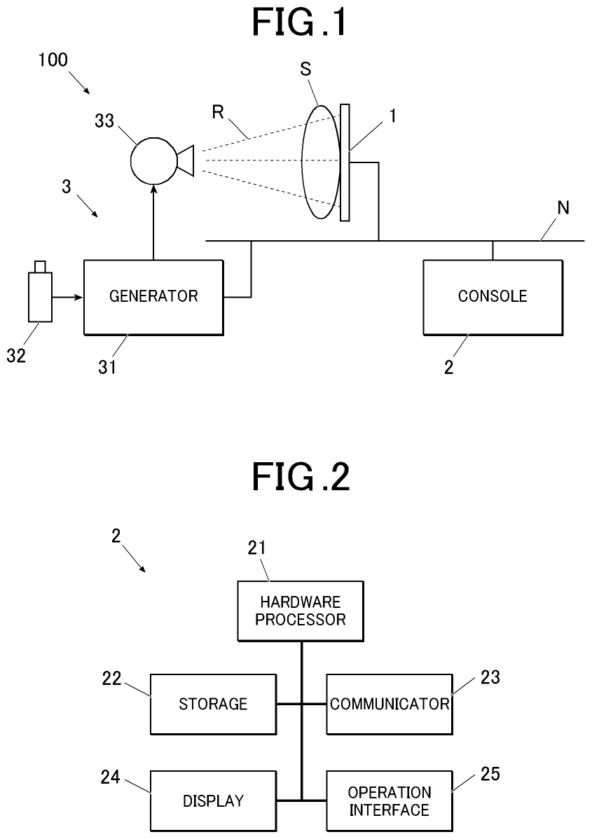

FIG. 1 is a block diagram showing the system 100.

The system 100 includes a plurality of types of devices for imaging used for dynamic imaging.

Specifically, for example, as shown in FIG. 1, the system 100 includes a radiation detector (hereinafter referred to as a detector 1) and a console 2.

In addition, the system 100 according to the present embodiment further includes a radiation generator (hereinafter referred to as a generator 3).

The devices 1 to 3 can communicate with each other via, for example, a communication network N (local area network (LAN), wide area network (WAN), the Internet, or the like).

The system 100 may be able to communicate with a hospital information system (HIS) (not shown), a radiology information system (RIS), a picture archiving and communication system (PACS), a dynamic analysis device, or the like.

In addition, the system 100 may be installed in an imaging room or may be a movable system called a round-trip car.

(1-1. Radiation Generator)

The generator 3 is a device for imaging that generates radiation R.

The generator 3 includes a generator 31, an irradiation instruction switch 32, and a tubular bulb 33.

The generator 31 applies a voltage according to imaging conditions (for example, conditions related to a subject (a person or an object, hereinafter referred to as a subject S) such as an imaging site, an imaging direction, and a physique and conditions related to the irradiation with the radiation R such as a tube voltage, a tube current, an irradiation time, and a tube current time product (mAs value)) preset based on the operation of the irradiation instruction switch 32 to the tubular bulb 33.

When the tube voltage is applied from the generator 31, the tubular bulb 33 generates the radiation R (for example, X-rays) having a dose corresponding to the applied tube voltage.

In addition, the tubular bulb 33 can move in an X-axis direction, a Y-axis direction orthogonal to the X-axis, and a Z-axis direction orthogonal to the X-axis and the Y-axis, and change the direction of an irradiation port of the radiation R by rotating around the rotation axis in parallel with the Y-axis and the Z-axis.

In addition, the generator 3 is allowed to generate the radiation R in a mode corresponding to the form of the radiation image (still image, dynamic image configured with a plurality of frames) to be generated.

In the case of a still image, the irradiation with the radiation R is performed only once for one time of pressing of the irradiation instruction switch 32.

In the case of a dynamic image, the irradiation with the pulsed radiation R is repeated several times per predetermined time (for example, 15 times per second) for one time of pressing of the irradiation instruction switch 32, or the irradiation with the radiation R is continued for a predetermined time.

(1-2. Radiation Detector)

The detector 1 is a device for imaging (image generator) that repeatedly generates frames constituting a dynamic image.

Although not shown, the detector 1 includes a sensor board in which pixels having scintillators emitting light with an intensity corresponding to a dose by receiving the radiation R, semiconductor elements generating electric charges according to an intensity of received light, or switch elements performing storing and discharging electric charges are arranged in a two-dimensional shape (matrix shape), a scanning circuit that switches on/off of each switch element, a readout circuit that reads out an amount of electric charges emitted from each pixel as a signal value, a hardware processor that generates a radiation image from a plurality of the signal values read out by the readout circuit, and a communicator that transmits data of the generated radiation image and various signals to the outside and receives various information and various signals.

The detector 1 stores and releases electric charges and reads out the signal value in synchronization with the timing when the radiation R is irradiated from the generator 3, so that the radiation image according to the dose distribution of the irradiated radiation R is generated.

In the case of generating a still image, the radiation image is generated only once for each pressing of the irradiation instruction switch 32.

In the case of generating a dynamic image, the generation of frames constituting the dynamic image is repeated a plurality of times per predetermined time (for example, 15 times per second) for each pressing of the irradiation instruction switch 32.

The detector 1 may not include a scintillator and may directly generate electric charges when the semiconductor element receives the radiation R.

In addition, the detector 1 may save and transfer the generated dynamic image in the form of image data, or may display the generated dynamic image on a display connected to the detector 1 itself in real time (for example, see through).

(1-3. Console)

The console 2 is a device for imaging that sets imaging conditions in at least one device of the detector 1 and the generator 3 based on imaging order information from an external device (RIS or the like) or an operation by a user.

The console 2 is configured with a PC, a mobile terminal, or a dedicated device.

In addition, the console 2 according to the present embodiment is allowed to execute a motion instruction process. That is, the console 2 according to the present embodiment has a motion instruction function.

Details of the console 2 including the motion instruction process will be described later.

(1-4. Others)

Heretofore, the system 100 including the console 2 having the motion instruction function has been described, but the motion instruction function may be possessed by the device for imaging (the detector 1 or the generator 3) other than the console 2.

In addition, the system 100 may include a device for imaging having a motion instruction function in addition to the detector 1, the console 2, and the generator 3.

[2. Details of Device for Imaging]

Next, the details of the device for imaging having the motion instruction function included in the system 100 will be described by taking the case of the console 2 as an example.

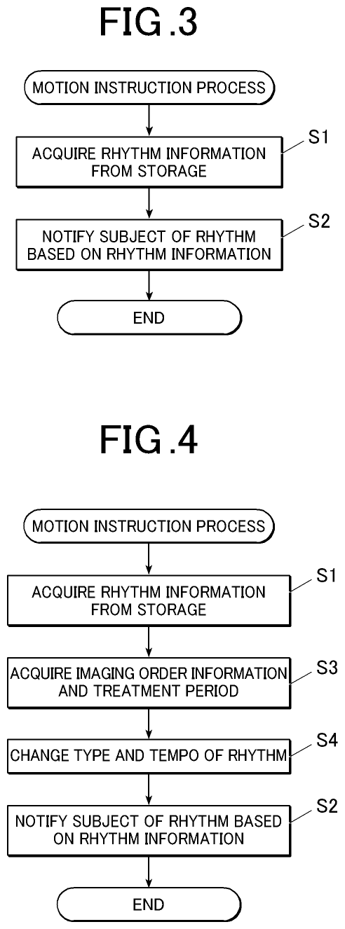

FIG. 2 is a block diagram showing the console 2, and FIGS. 3 and 4 are flowcharts showing a flow of a motion instruction process executed by the console 2.

(2-1. Configuration of Device for Imaging)

As shown in FIG. 2, the console 2 also serving as a device for imaging includes a hardware processor 21, a storage 22, a communicator 23, a display 24, and an operation interface 25.

Each component 21 to 25 is electrically connected by a bus or the like.

The hardware processor 21 is configured with a central processing unit (CPU), a random access memory (RAM), and the like.

The CPU of the hardware processor 21 reads various programs stored in the storage 22 to load the programs in the RAM, executes various processes according to the loaded programs, and centrally controls the operation of each component of the console 2.

The storage 22 is configured with a non-volatile memory, a hard disk, and the like.

In addition, the storage 22 stores various programs executed by the hardware processor 21, parameters necessary for executing the programs, and the like.

The storage 22 may be capable of storing image data of a radiation image acquired from another device.

In addition, the storage 22 also serves as a storage for storing the rhythm information.

The rhythm information defines a rhythm for the subject S to recognize a start timing, a switch timing, or a stop timing of a predetermined periodic motion.

The rhythm information stored in the storage 22 may be of one type or may be of a plurality of types.

The communicator 23 is configured with a communication module and the like.

The communicator 23 is connected to another device (the detector 1, the generator 3, or the like) by wire or wirelessly via a communication network N (local area network (LAN), wide area network (WAN), the Internet, or the like) to transmit and receive various signals, various data, or the like.

The display 24 is configured with, for example, a liquid crystal display (LCD), a cathode ray tube (CRT), or the like.

The display 24 is allowed to display a radiation image or the like corresponding to the image signal received from the hardware processor 21.

The operation interface 25 is an operation interface that can be operated by the user.

The operation interface 25 includes a keyboard (a cursor key, number input keys, various function keys, and the like), a pointing device (mouse or the like), a touch panel stacked on the surface of the display 24, and the like.

The operation interface 25 outputs a control signal corresponding to the operation performed by the user to the hardware processor 21.

The console 2 does not include the display 24 and the operation interface 25, and the console 2 may receive a control signal from an input device provided separately from the console 2 via, for example, the communicator 23 or the like, or may output an image signal to a display (monitor) provided separately from the console 2.

In addition, in a case where another device (generator device 3 or the like) is provided with a display and an operation interface, a control signal may be allowed to be received from the operation interface of the other device, or an image signal may be allowed to be output to the display of the other device (the display and the operation interface may be shared with other device).

(2-2. Operation of Device for Imaging)

The hardware processor 21 of the console 2 configured as described above executes, for example, the motion instruction process as shown in FIG. 3, when a predetermined condition is satisfied.

The predetermined conditions include, for example, receiving a predetermined control signal from another device, performing a predetermined operation on the operation interface 25, and the like.

In this motion instruction process, the hardware processor 21 first executes an acquisition process (step S1).

In the acquisition process, the hardware processor 21 acquires rhythm information from the storage 22 (storage).

In the acquisition process, the hardware processor 21 may read the rhythm information from a storage medium (a USB memory, an SD card, and the like) (not shown).

By executing the acquisition process described above, the hardware processor 21 serves as an acquisitor.

After acquiring the rhythm information, the hardware processor 21 executes a notification process (step S2).

In the notification process, the hardware processor 21 notifies the subject S of the rhythm based on the acquired rhythm information.

The hardware processor 21 according to the present embodiment notifies the subject S of the rhythm through at least one of the visual, auditory, and tactile senses of the subject S.

Specifically, the hardware processor 21 performs at least one of operations of allowing the display 24 and the like to display an image, allowing the speaker to output sound, and allowing the vibrating device to physically vibrate.

In particular, if the rhythm is notified to the subject S through a combination of at least two senses of the visual, auditory, and tactile senses, a synergistic effect of these can be expected.

The image to be displayed on the display 24 or the like may be, for example, an image obtained by repeating display/non-display of one image, or an image obtained by repeating switching display of two or more images.

A device other than the display 24 may display the image.

In addition, the rhythm may be notified by a method such as allowing a lamp to emit light instead of displaying an image.

The sound output to the speaker may be, for example, a repetition of a simple (same pitch) sound such as that output by a metronome or may be a repetition of switching between a plurality of types of sounds having different pitches.

Each note may be a single note or a chord.

The voice output to the speaker may be a simple language (for example, a number) that is not directly linked to the motion.

Specifically, it may be a voice that counts up as “1, 2, 3, 4 . . . ” or counts down as “6, 5, 4, 3 . . . ”, a voice that repeats “1, 2, 1, 2 . . . ”, and the like.

The vibrating device that physically vibrates may be a device that vibrates mechanically or may be a device that vibrates electrically.

In addition, the vibrating device may be a device installed in the imaging room or a wearable device worn by the subject S.

In addition, the hardware processor 21 notifies the subject S of the rhythm before starting generation of a frame and during at least one of several periods of repeating the generation of the frame.

“Before starting generation of a frame” includes, for example, a period for pre-practicing the motion when performing the dynamic imaging.

In addition, “several periods of repeating the generation of the frame” includes a period during which the dynamic imaging is actually performed.

In the notification process, the hardware processor 21 may notify a rhythm of which tempo is not constant.

Specifically, for example, the hardware processor 21 may perform the notification so that the interval between the notifications gradually becomes shorter as time elapses, and conversely, the hardware processor 21 may perform the notification so that the interval between the notifications gradually becomes longer.

By executing the notification process described above, at least one of the hardware processor 21, the display 24, the speaker, and the vibration device serves as a notificator.

(2-3. Others)

In a case where a plurality of types of the rhythm information are stored in the storage 22, the hardware processor 21 may use the rhythm information to be used properly according to the situation of the subject S.

The “situation” includes, for example, age, symptoms, and the like.

For example, in the case of a subject S of which age is a predetermined age or older, since it is considered that it is difficult to perceive the rhythm, the hardware processor 21 selects rhythm information that the subject S can more easily perceive.

In addition, in the case of a subject S having a severe symptom, since it is considered that the time from perceiving the rhythm to starting the motion may be long, the hardware processor 21 selects the rhythm information having a slow tempo.

In addition, in the motion instruction process, the hardware processor 21 may further execute a second acquisition process and a change process, for example, as shown in FIG. 4 (steps S3 and S4).

In this case, in the second acquisition process, the hardware processor 21 acquires at least one of the imaging order information and the treatment period.

The imaging order information includes information on at least one of the current imaging and the past imaging.

In addition, the past imaging order information includes at least one of the imaging order information in a case where the subject S is the same as the subject S of the current imaging and the imaging order information in the case of another subject S who is different from the subject S of the current imaging but has similar symptoms.

In addition, in the change process, the hardware processor 21 changes at least one of the type and the tempo of the rhythm based on the information acquired in the second acquisition process.

For example, when the contents of the acquired past imaging order information are the same as the contents of the current imaging order information, the hardware processor 21 may set the rhythm and tempo notified in the current dynamic imaging to be the same as those in the past dynamic imaging.

In addition, when the treatment period is longer than a predetermined length, it is considered that the recovery of the symptom is progressing, so that the hardware processor 21 may perform controlling such as increasing the tempo of the rhythm to be faster than the previous time.

In addition, in the second acquisition process, the hardware processor 21 may acquire data of past dynamic image.

In this case, in the change process, the hardware processor 21 may change at least one of the type and the tempo of the rhythm based on the acquired date of the dynamic image.

In addition, in this case, the tendency (recovery, deterioration, or the like) of the symptom of the subject S is estimated from a plurality of the past dynamic images of the same subject S, and at least one of the type and tempo of the rhythm may be changed based on an estimation result.

For example, in a case where the movable range of the joint shown in the dynamic image obtained relatively later is larger than the movable range shown in the dynamic image obtained relatively earlier, the hardware processor 21 determines that the symptom of the subject S is in a recovering tendency and performs controlling such as increasing the tempo of the rhythm.

If the second acquisition process and the change process described above are executed, the hardware processor 21 serves as a second acquisitor and a changer, and thus, the labor of the user performing changing the type and tempo of the rhythm can be omitted.

In addition, when the system 100 can perform a plurality of types of notifications having different notification modes, the hardware processor 21 may further execute a selection process in the motion instruction process.

In this case, in the selection process, the notification mode of the rhythm is selected based on the operation performed by the user on the operation interface 25.

In addition, in this case, in the notification process, the hardware processor 21 notifies the subject S of the rhythm in the selected notification mode.

If the selection process described above is executed, the hardware processor 21 serves as a selector, and the rhythm can be notified to the user in the notification mode according to the user's request.

In addition, in the motion instruction process, the hardware processor 21 may further execute an adjustment process.

In this case, in the adjustment process, the hardware processor 21 adjusts the tempo of the rhythm based on the operation performed by the user on the operation interface 25.

If the adjustment process described above is executed, the hardware processor 21 serves as an adjuster, and the rhythm can be notified to the user at a tempo according to the user's request.

In addition, when the hardware processor 21 completes executing the motion instruction process, the detector 1 generates a dynamic image.

Therefore, after executing the motion instruction process, the hardware processor 21 may associate the data of the dynamic image generated by the detector 1 with the rhythm information used when executing the motion instruction process corresponding to the dynamic image or the contents of the actual notification.

By doing so, it is possible to easily know what kind of rhythm is notified to obtain the dynamic image.

In addition, in the motion instruction process, the hardware processor 21 may start the notification process based on the operation of the irradiation instruction switch 32.

[3. Flow of Dynamic Imaging]

Next, regarding the flow of the dynamic imaging by using the system 100, a case where the bending and stretching of the neck is imaged by notifying the rhythm by counting up the numbers will be described.

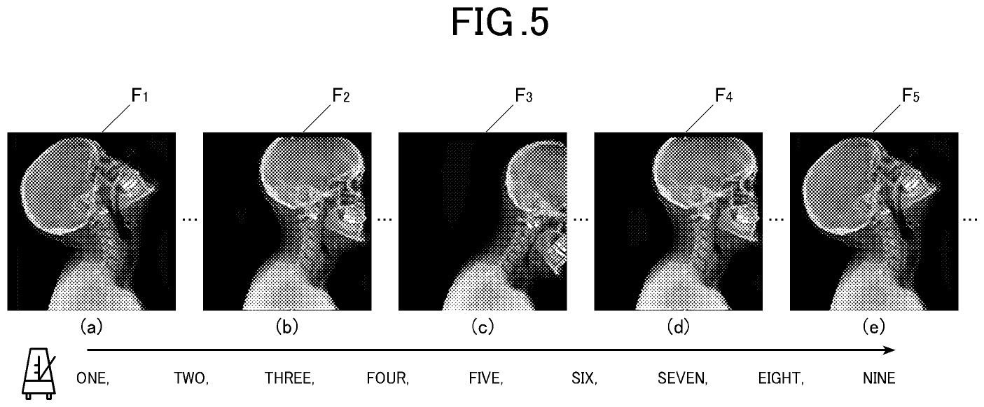

FIG. 5 is a diagram showing the correspondence between the operations of the device for imaging 2 and the generated frame.

In order to perform the dynamic imaging by using the system 100, first, a method of motion during the dynamic imaging will be explained to the subject S.

For example, the subject S is notified of what kind of notification is given during of imaging (for example, the console 2 is actually moved to notify the rhythm) before the start of imaging.

Then, when the first rhythm (“1”) is notified, the subject S is informed that the subject S will take a first posture (for example, the state in which the neck is bent backward).

Next, the subject S is informed that the subject S will take a second posture (for example, a state in which the neck is extended) until the rhythm (herein, “3”) after a predetermined number of times from the first rhythm is notified.

Next, the subject S is informed that the subject S will take a third posture (for example, a state in which the neck is bent forward) until the rhythm (herein, “5”) after a predetermined number of times from the time when the second posture is completed is notified.

Next, the subject S is informed that the subject S will take a fourth posture (for example, a state in which the neck is extended) until the rhythm (herein, “7”) after a predetermined number of times from the time when the third posture is completed is notified.

After that, when a predetermined number of rhythms (herein, “9”, “11” . . . ) is notified, the subject S is informed that the switching of the first to fourth postures is repeated.

After completing the explanation, the process proceeds to dynamic imaging.

In the dynamic imaging, the device for imaging notifies the subject S of a monotonous rhythm (for example, counting up numbers). That is, the notifications such as “forward, forward, forward” and “yes, return” in the related art are not performed.

When the first rhythm is notified, the subject S takes a first posture. Then, as shown in FIG. 5, the detector 1 generates a plurality of frames in which the subject S is captured until the first posture is taken (including a frame F₁ in which the subject S in the first posture is captured).

When the rhythm after a predetermined number of times is notified from the first rhythm, the subject S takes a second posture. Then, the detector 1 generates a plurality of frames (including a frame F₂ in which the subject S in the second posture is captured) in which the subject S is captured until the second posture is taken.

When the rhythm is notified a predetermined number of times after the second posture is completed, the subject S takes a third posture. Then, the detector 1 generates a plurality of frames (including a frame F₃ in which the subject S in the third posture is captured) in which the subject S is captured until the third posture is taken.

When the rhythm after a predetermined number of times is notified after the third posture is completed, the subject S takes a fourth posture. Then, the detector 1 generates a plurality of frames (including a frame F4 in which the subject S in the fourth posture is captured) in which the subject S is captured until the fourth posture is taken.

After that, when the rhythm is notified a predetermined number of times, the subject S repeats switching of the first to fourth postures. The detector 1 repeats the generation of the frame in which the subject S of which posture is switched is captured.

In this manner, by notifying the monotonous rhythm, the subject S can easily grasp the timing at which the motion switching is started, such as by taking a beat by himself or herself.

In addition, when the subject S grasps the rhythm, even if the subject S misses or overlooks the notification of the rhythm during the imaging, the motion of the subject S is less likely to be disturbed, and even if the motion is disturbed, it will be easier to recover to the original motion.

As a result, the possibility of failure of dynamic imaging can be reduced.

[4. Function and Effect]

As described above, the console 2 (device for imaging) according to the present embodiment or the system 100 including the console 2 is allowed to execute an acquisition process of acquiring rhythm information from a storage for storing the rhythm information that defines a rhythm in order to cause a subject S to recognize a start timing, a switch timing, or a stop timing of a predetermined periodic motion and a notification process of notifying the subject S of the rhythm based on the rhythm information acquired in the acquisition process before starting generation of a frame and during at least one of several periods of repeating the generation of the frame.

Unlike the imaging guide pattern in the related art, the rhythm is not a language, so that, no matter what the periodic motion tries to be imaged, the subject S can be instructed to start, switch, or stop of the motion just by notifying the common rhythm (versatility is high).

In addition, since the rhythm information is limited to one or several types, a complicated selection operation by the user becomes unnecessary.

In addition, since it is not necessary to add data, it is possible to reduce the labor of the serviceman, and there is no problem that the data capacity of the storage 22 becomes tight and the motion instruction process becomes complicated.

For this reason, according to the console 2 or the system 100, in the dynamic imaging which is performed while instructing the subject to start, switch, or stop a predetermined periodic motion, instruction of a plurality of periodic motions having different contents is enabled to be performed without a device configuration (stored data, programs, and the like) and an operation by a user being complicated.

[5. Others]

Needless to say, the invention is not limited to the above-described embodiments and the like, and can be appropriately modified without departing from the spirit of the invention.

For example, in the above description, the example in which a hard disk, a semiconductor non-volatile memory, or the like is used as a computer-readable medium for the program according to the invention has been disclosed, but the invention is not limited to this example. As another computer-readable medium, a portable recording medium such as a CD-ROM can be applied. In addition, a carrier wave is also applied as a medium for providing data of the program according to the invention via a communication line.

Although embodiments of the present invention have been described and illustrated in detail, the disclosed embodiments are made for purposes of illustration and example only and not limitation. The scope of the present invention should be interpreted by terms of the appended claims.

What is claimed is:

1. A device for imaging used for dynamic imaging that repeatedly generates a plurality of frames, comprising a hardware processor,
wherein the hardware processor acquires rhythm information from a storage for storing the rhythm information that defines a rhythm in order to cause a subject to recognize a start timing, a switch timing, or a stop timing of a predetermined periodic motion, and
wherein the hardware processor notifies the subject of the rhythm based on the acquired rhythm information at least one of periods before starting generation of a frame and during repeating of the generation of the frame.

2. The device for imaging according to claim 1, wherein the hardware processor notifies the subject of the rhythm through at least one sense of a visual sense, an auditory sense, and a tactile sense of the subject.

3. The device for imaging according to claim 2, further comprising a user-operable operation interface,
wherein the hardware processor is capable of performing a plurality of types of notifications having different notification modes,
wherein one of the notification modes is selected based on an operation performed by a user on the operation interface, and
wherein the rhythm is notified to the subject in the selected notification mode.

4. The device for imaging according to claim 2, wherein the hardware processor notifies the subject of the rhythm through a combination of at least two senses of the visual sense, the auditory sense, and the tactile sense.

5. The device for imaging according to claim 1, further comprising a user-operable operation interface, wherein the hardware processor adjusts a tempo of the rhythm based on the operation performed by the user on the operation interface.

6. The device for imaging according to claim 1,
wherein the hardware processor acquires information of at least one of imaging order information and a treatment period, and
wherein at least one of the type and tempo of the rhythm is changed based on the information of at least one of the acquired imaging order information and the treatment period.

7. A radiation imaging system comprising:
a storage that stores rhythm information that defines a rhythm for a subject to recognize a start timing, a switch timing, or a stop timing of a predetermined periodic motion;
an image generator that repeatedly generates frames constituting dynamic image; and
a hardware processor that acquires the rhythm information from the storage and that notifies the subject of the rhythm based on the rhythm information that has been acquired at least one of periods before starting generation of a frame and during repeating of the generation of the frame by the image generator.

8. A computer-readable non-temporary recording medium containing a program, wherein the program causes a hardware processor of a device for imaging used for dynamic imaging that repeatedly generates a plurality of frames to execute:
an acquisition process of acquiring rhythm information from a storage for storing the rhythm information that defines a rhythm in order to cause a subject to recognize a start timing, a switch timing, or a stop timing of a predetermined periodic motion; and
a notification process for notifying the subject of the rhythm based on the rhythm information acquired in the acquisition process before starting generation of a frame and during at least one of several periods of repeating the generation of the frame.
